(19) United States
(12) Patent Application Publication
(10) Pub. No.: US 2004/0027538 A1
(43) Pub. Date: Feb. 12, 2004

(54) System and method for testing visual capability and for assessing ocular toxicity and ocular effects of bio-effective materials

(76) Inventor: Stephen Sinclair, Gladwyn, PA (US)

Correspondence Address: Lawrence A. Husick, Esq., Lipton, Weinberger & Husick, 201 North Jackson Street, Media, PA, 19063, US

(21) Appl. No.: 10/215,363

(22) Filed: Aug. 8, 2002

(51) Int. Cl. A61B 3/02

(52) U.S. Cl. 351/224

(57) ABSTRACT

An automated system for assessing the visual capability of a human subject under a wide range of optical environmental conditions including low illumination, glare, low and high contrast, and off axis events, which, used over time during the administration of a bio-effective compound, permits even subtle changes in visual capability of a subject to be detected.

### FIELD OF THE INVENTION

The present invention pertains to automated systems for assessing the visual capability of a human subject under a wide range of optical environmental conditions including low illumination, glare, low and high contrast, and events off the central axis of the field of vision. Used over time during the administration of a bio-effective compound, even subtle changes in visual capability of a subject may be detected.

### BACKGROUND OF THE INVENTION

Methods of assessing acuity at the axis of fixation, such as the Snellen or Lovie-Bailley charts have been used for almost 150 years in order to determine the relative performance of a human subject's vision. Results of these tests are reported as a fraction such as 20/20, where the numerator represents the 20 foot visual acuity of the human subject, and the denominator represents the distance at which a normal human subject is also able to discern the same letter size. Thus, 20/200 is interpreted to mean that what the test subject sees at 20 feet, an average person can distinguish at 200 feet from the object. Conversely, 20/10 is interpreted to mean that the subject has hyper-acuity; as what a normal person sees at ten feet, the subject sees at 20. This system only measures high contrast acuity without color evaluation, and only in the central one degree of vision under one illumination condition that is not controlled. It is insensitive to subtle changes in the refracting medium or retinal or neural perception. Similarly, contrast sensitivity using Nyquist sinusoidal grating measures non-discriminative targets in the central four degrees of vision under one illumination level.

U.S. Pat. No. 5,589,897, entitled “Method and apparatus for central visual field mapping and optimization of image presentation based upon mapped parameters” teaches a method and apparatus provides a much more precise and accurate measurement of a human subjects visual capability across a wide region of the visual field. The disclosure and claims of that patent are incorporated herein by reference.

It is generally known that many bio-effective compounds induce changes in visual capability. In the medical literature, for example, the compound chloroquine was shown to be toxic to the retina, although this side effect of the drug was not well understood until after many patients had lost their sight. Because of such effects, the United States Food and Drug Administration requires that all new drug candidates be screened for clinical evidence of ocular toxicity. Methods for such screening, including the central chart acuity, contrast sensitivity, and color vision testing (Farnsworth DI5 panel), however, are often inadequate, and fail to quantify subtle changes occurring over long time periods, in perception areas off the central axis. Humphrey automated field testing is not commonly used, nor does it utilize discriminated targets and tests only under one level of illumination.

### BRIEF DESCRIPTION OF THE INVENTION

The present invention comprises in combination, a general purpose digital computer system having a display screen and a directional user input device, and special purpose software for displaying visual stimuli and recording user response input about them. This system is employed over time to provide a longitudinal record of the visual capabilities of a subject, and to allow recognition of changes and trends in such visual capabilities.

### DETAILED DESCRIPTION OF THE INVENTION

Visual acuity is usually tested in the central visual field by testing the ability of a human subject to discern a variety of letters on a wall chart, mounted at a standard distance from the subject, with standard illumination. Such tests, therefore, require the services of a technician to listen to the test subject and interpret his/her responses in a non-standardized method that varies with response and technician interpretation. In the system of the present invention, the letter “C” is presented in one of four orientations (facing with the open segment up, down, left or right) and the direction of the orientation is indicated by the test subject using a directional input device connected to the computer system (for instance, a common game pad having a diamond-shaped array of four button switches.) In the present system, the letter “C” is presented in progressively larger and smaller sizes, and varying levels of luminance and in varying combinations of hue and saturation, against backgrounds that are also varied in luminance, hue, and saturation. In this way, visual acuity (ability to discriminate targets at central fixation) may be measured in many dimensions such as spatial acuity, contrast acuity (luminance differential under conditions of low, normal, and high illumination), color contrast acuity (ability to distinguish among various hues), and color sensitivity (ability to distinguish among various saturations of the same or similar hues.) In addition, when using a fixation target, the ability of the test subject may be optionally monitored using an optical fixation system (such as that available from Iscan, Inc.), and more than just the central visual field may be tested. Typically, the central field can be measured at multiple intercepts (25 to 90) in the central twenty degrees of vision.

The system of the present invention is employed before administration of a bio-effective compound to test the subject and to establish a baseline of visual capability for that subject. Thereafter, the bio-effective compound is administered, and after one or more intervals, the subject is re-tested using the system of the present invention, and the results compared with one or more previous results. Changes and trends may thus be presented and analyzed.

While the invention has been described in its preferred embodiments, it is to be understood that the words which have been used are words of description rather than of limitation and that changes may be made within the purview of the appended claims without departing from the true scope and spirit of the invention in its broader aspects. The inventor further requires that the scope accorded his claims be in accordance with the broadest possible construction available under the law as it exists on the date of filing hereof, and that no narrowing of the scope of the appended claims be allowed due to subsequent changes in the law, as such a narrowing would constitute an ex post facto law, and a taking without due process or just compensation.

1. A method for longitudinal assessment of ocular toxicity of a compound to which a human subject is exposed comprising the steps of:
a. Testing at least the central visual capability of the human subject prior to exposure to the compound, wherein the test assesses the subject's capability under conditions of high glare, low illumination, high contrast, low contrast, color contrast and saturation contrast;
b. Testing at least the central visual capability of the human subject periodically during and after exposure to the compound; and
c. Comparing the results of the at least two of the tests to determine whether the subject's visual capability has been affected.

2. The method of claim 1 wherein the testing of visual capability includes a region of at least 5 degrees outside of the central visual field.

3. The method of claim 1 wherein the testing of visual capability includes a region of at least 10 degrees outside of the central visual field.

4. The method of claim 1 wherein the testing of visual capability comprises the exposure of the human subject to a visual display of a plurality of differently oriented directional figures, and the human subject is required to indicate the directional orientation of each of the figures by direct input to a digital computer.

5. The method of claim 4 wherein the directional figures are presented in a plurality of sizes.

6. The method of claim 4 wherein the directional figures are presented in one of a plurality of hues, each against a background having one of a plurality of different hues.

7. The method of claim 4 wherein the directional figures are presented in one of a plurality of saturations of one of a plurality of hues, each against a background having a different saturation of the same hue as the figure.

8. The method of claim 1 wherein the comparison of results comprises a comparison of each of at least a pair of measurements of visual ability of the human subject, to detect changes therein.

9. The method of claim 8 wherein the comparison also results in the presentation of trend indications about each of the pairs of measurements compared.

10. A system for longitudinal assessment of ocular toxicity of a compound to which a human subject is exposed comprising:
a. Means for testing the visual capability of the human subject at fixation and at a plurality of intercepts angularly distant from fixation, prior to exposure to the compound, wherein the test assesses the subject's capability under conditions selected from the group of low illumination, moderate illumination and glare illumination, high contrast, low contrast, color contrast and saturation contrast;
b. Means for storing at least two results of tests conducted using the means (a); and
c. Means for comparing the results of the at least two of the stored tests to determine whether the subject's visual capability has been affected.
